(19) United States
(12) Patent Application Publication
(10) Pub. No.: US 2007/0116768 A1
(43) Pub. Date: May 24, 2007

(54) Sustained release preparations composed of biocompatible complex microparticles

(76) Inventors: Michael Chorny, Philadelphia, PA (US); Robert J. Levy, Merion Station, PA (US)

Correspondence Address: RATNERPRESTIA, P O BOX 980, VALLEY FORGE, PA, 19482-0980, US

(21) Appl. No.: 10/582,266

(22) PCT Filed: Dec. 9, 2004
(86) PCT No.: PCT/US04/41058

§ 371 Date: Jun. 9, 2006

Related U.S. Application Data
(60) Provisional application No. 60/528,458, filed on Dec. 9, 2003.

(51) Int. Cl. A61K 39/395; A61K 38/22; A61K 38/18; A61K 31/715; A61K 9/14

(52) U.S. Cl. 424/489; 514/12; 514/54; 424/133.1

(57) ABSTRACT

A particle includes a complex between a bioactive agent and a complexing agent, provided that the bioactive agent is other than a polynucleotide and an oligonucleotide, and wherein the particle has a bioactive function. The particle has a diameter from about 5 nm to about 100 microns. In certain embodiments, the bioactive agent is a member selected from the group consisting of a growth factor, a hormone, a peptide, a protein, and polysaccharide. In certain embodiments, the complexing agent is a member selected from the group consisting of polysaccharides, glycosaminoglycans, complex carbohydrates, polyacids, modifications and derivatives thereof. Also provided is a method of making the particle. Further provided is a method of administering the particle, including providing the particle, which is adapted to gradually release the bioactive agent; and thereby administering the particle.

### CROSS-REFERENCE TO RELATED APPLICATIONS

This application claims the benefit of provisional Application No. 60/528,458 filed on Dec. 9, 2003, which is incorporated herein in its entirety.

### SPECIFICATION

### BACKGROUND OF THE INVENTION

1. Field of Invention

This invention relates to microparticle and nanoparticle compositions, methods of making and administration thereof and more particularly it relates to ionically formed biologically active microparticle and nanoparticle compositions capable of sustained release of biomolecules.

2. Description of Related Art

The main focus of research in the area of drug delivery systems has been on providing for controlled release of bioactive agents and acceptable concentration of such agents. An extensive research has been conducted in the area of coupling a biomolecule with a biodegradable polymer, wherein the biomolecule is released upon diffusion out or degradation of the polymer. Methods of delivery of the biomaterial to an organism are also an important factor in the design of drug delivery systems.

Heparin-binding growth factors have been studied for ways to improve bioavailability. Studies of interactions of a polyanionic molecule with vascular endothelial growth factor (VEGF) and human growth factor (HGF) demonstrated potential for extending the half life of the growth factors, preventing their deactivation in the biological milieu and improving their bioavailability following injection by co-administering with a polyanionic molecule such as heparin or heparin derived polysaccharides. (See U.S. Pat. No. 5,851,989 to Chamow et al. and U.S. Pat. No. 5,736,506 to Naka). Growth factors were not administered as particle systems/complexes.

Further, it has been shown that dextran sulfate does not suppress platelet-derived growth factor (PDGF)-stimulated cell proliferation in fibroblast culture (See Powis G, Seewald M, Hoke M, Inhibition of growth factor binding and intracellular Ca2+ signaling by dextran sulfates of different sizes and degrees of sulfation. Cancer Chemother Pharmacol. 1992; 30(6):483-6).

A sustained release composition (but not as injectable microparticles) employing covalent binding of dextran and IGF-1 or hGH has also been described ( See U.S. Pat. No. 5,614,487 to Battersby et al.). Battersby et al., disclose sustained release pharmaceutical compositions comprising imine adducts of biologically active peptide, e.g., human growth hormone and dextran. These adducts serve to release the biologically active peptide via the reversible reaction in the formation of the imine adduct bond.

U.S. Pat. No.4,107,288 to Oppenheim et al. discloses injectable nanoparticles andthe process of making thereof, wherein the nanoparticles comprise a biologically active material supported on or incorporated into a crosslinked matrix.

U.S. Pat. No. 4,634,762 to Feijen et al. discloses covalently bonded conjugates of protein and anticoagulant (heparin) prepared in the presence of a coupling agent.

U.S. Pat. No.6,645,525 B1 to Woiszwillo and an article by Waree Tiyaboonchai et al. (Insulin Containing Polyethylenimine-Dextran Sulfate Nanoparticles; International Journal of Pharmaceutics, 225 (2003) 139-151) disclose compositions for sustained delivery of a biomolecule including an anionic and a cationic polymers that ionically interact with each other and optionally with the biomolecule. In this disclosure, the anionic polymer and the cationic polymer form a microparticle and the biomolecule interacts with one of the polymers before it is complexed with the oppositely charged polymer. Examples include insulin complexed with dextran sulfate. Next, the insulin-dextran sulfate complex is combined with the oppositely charged polymer and a precipitating agent to form a microparticle. In this disclosure, the biomolecule does not play a structural role in forming the microparticle, but the anionic polymer and the cationic polymer. Also, the initiator such as zinc sulfate is required for forming microparticles (microcarriers).

An article by Enghild et al. discloses that the heparin-binding domain of EC-SOD contains six positively charged amino acids (Arg-Lys-Lys-Arg-Arg-Arg) (The Heparin-binding Domain of Extracellular Superoxide Dismutase is Proteolytically Processed Intracellularly During Biosynthesis; The Journal of Biological Chemistry, Vol. 274, No. 21, Issue of May 21, pp. 14818-14822, 1999).

An article by Schilling et al. discloses that the cationic residues Arg 159, Lys 160, and Lys 161 of the A-chain could be considered a heparin binding region for PDFG (Loop III Region of Platelet-Derived Growth Factor (PDGF) B-Chain Mediates Binding to PDGF Receptors and Heparin; Biochem J. (1998) 333, 637-644). PDGF is a family of three disulphide-linked glycoprotein dimers of molecular masses 29-32 KDa that arise from stochastic assembly of the homologous subunits, A-chain and B-chain, yielding the heterodimer PDGF-AB and homodimers PDGF-AA and PDGF-BB.

Fairbrother et al. studied heparin-binding domains of VEGF and mutants thereof (Solution Structure of the Heparin-Binding Domain of Vascular Endothelial Growth Factor; Structure, 15 May 1998, 6:637-648). The 55-residue heparin-binding domain of VEGF₁₆₅ is disclosed.

An article by Turgeon et al. discusses mechanisms of protein-polysaccharide interactions (Protein-Polysaccharide Interactions: Phase-Ordering Kinetics, Thermodynamic and Structural Aspects; Current Opinion in Colloid and Interface Science 8 (2003) 401-414). One of the mechanisms is a thermodynamic incompatibility, which is predominantly entropically driven. Another mechanism is a complex coacervation, which is both entropically and enthalpically driven. It is noted that electrostatic interactions play an important role in case of the complex coacervation wherein the following factors such as pH, a protein-polysaccharide ratio, and ionic strength are important parameters for stability of the complex. However, other non-specific interactions such as hydrogen bonding or hydrophobic interactions also influence formation of the protein/polysaccharide complex. It was also noted that complexation between a polyelectrolyte and a protein may still occur when both macromolecules are negatively charged.

Several studies describe preparing complexes between polyanions and proteins. (See Interpolyelectrolyte Complex and Coacervate Formation of Poly(glutamic acid) with a Dendrimer Studied by Light Scattering and SAXS; Leisner et al., J. Phys. Chem. B 2003, 107, 8078-8087; Influence of Complexing Polyanions on the Thermostability of Basic Proteins; Ivinova et al., Macromol. Biosci 2003, 3, 210-215; Effect of Polysaccharides on the Stability and Renaturation of Soybean Trypsin (Kunitz) Inhibitor; Burova et al., Macromol. Biosci., 2002, 2, 286-292).

A complex coacervation procedure is described by Wang et al., Protein Separation via Polyelectrolyte Coacervation: Selectivity and Efficiency. Biotechnol Prog. 1996; 12: 356-362.

Sustained release preparations containing peptides and proteins have a broad field of use in medicine, for example, for diabetes (insulin), bone regeneration (BMP's), cancer therapy (Fas-L), and angiogenesis (VEGF, PDGF). Problems with the currently available drug delivery complexes include the necessity of using stringent conditions and solvents, as well as multiple steps for forming particles. Thus, proteins and peptides undergo structural and functional damage when incorporated into sustained release preparations.

Despite the foregoing developments, there is a need in the art for drug delivery particle complexes which avoid the problems discussed above.

All references cited herein are incorporated herein by reference in their entireties.

### BRIEF SUMMARY OF THE INVENTION

Accordingly, the invention provides a particle comprising a complex between a bioactive agent and a complexing agent, provided that the bioactive agent is other than a polynucleotide and an oligonucleotide, and wherein the particle has a bioactive function conferred to the particle by the bioactive agent.

In certain embodiments, the bioactive agent and/or the complexing agent have a net positive charge or have a positively charged region of at least +6.

In certain embodiments, the bioactive agent has the net positive charge or the positively charged region of at least +6 and the complexing agent has the net negative charge or the negatively charged region of at least −6.

In certain embodiments, the net positive charge is conferred by at least six aminoacids selected from the group consisting of lysine, arginine, and histidine.

In certain embodiments, the positively charged region is a heparin binding domain.

In certain embodiments, the other of the bioactive agent and the complexing agent has a net negative charge or has a negatively charged region of at least −6.

In certain embodiments, the particle has a diameter from about 5 nm to about 100 microns.

In certain embodiments, the bioactive agent is a member selected from the group consisting of a growth factor, a hormone, a peptide, a protein, and polysaccharide.

In certain embodiments, the bioactive agent is a growth factor.

In certain embodiments, the growth factor is VEGF, PDGF, FGF, bFGF, or HGH.

In certain embodiments, the bioactive agent is at least one of VEGF and PDGF.

In certain embodiments, the bioactive agent is a member selected from the group consisting of insulin, erythropoietin, bone morphogenic proteins, human chorionic gonadotrophin, chitosan, transferrin, TGF-beta receptors, integrin heterodimer receptor, receptor fragments for FGF, PDGF, VEGF, and CAR.

In certain embodiments, the complexing agent is a member selected from the group consisting of polysaccharides, glycosaminoglycans, complex carbohydrates, polyacids, modifications and derivatives thereof.

In certain embodiments, the complexing agent is a member selected from the group consisting of dextran, dextran sulfate, chitosan, heparin, heparan, heparan sulfate, hyaluronic acid, chondroitin, chondroitin sulfate, dermatan sufate, keratan sulfate, pentasan sulfate, alginate, and carageenan, polyglutamic acid, and 3-polyphosphoric acid.

In certain embodiments, the complexing agent is dextran or dextran sulfate having a molecular weight of about 2 KDa to about 10,000 KDa.

In certain embodiments, the molecular weight of dextran or dextran sulfate is from 5 KDa to 500 KDa

In certain embodiments, the particle is free of poly(ethyleneimine).

In certain embodiments, the particle of the invention further comprises an agent, wherein the agent is a member selected from the group consisting of an antibody, an antigen, a receptor, and a ligand.

In certain embodiments, the particle of the invention futher comprises a matrix, wherein the matrix is associated with at least one of the bioactive agent and the complexing agent.

In certain embodiments, the matrix is a member selected from the group consisting of biodegradable polymers, colloidal particles, liposomes, emulsions, solid particles, magnetic particles, proteins, and peptides.

In certain embodiments, the particle is completely biodegradable.

In certain embodiments, the particle of claim 1, wherein the particle comprises at least 40% of the bioactive agent.

In certain embodiments, the particle comprises from about 40% to about 90% of the bioactive agent.

In certain embodiments, the particle consists essentially of the bioactive agent and the complexing agent.

Further provided is a particle comprising a complex between a bioactive agent and a complexing agent, wherein one of the bioactive agent and the complexing agent is a cationic agent or an anionic agent having a net charge or a region having a net charge of at least 6 units.

In certain embodiments, the bioactive agent is the cationic agent and the complexing agent is the anionic agent.

In certain embodiments, the cationic agent is a member selected from the group consisting of a growth factor, a hormone, a peptide, a protein, and polysaccharide.

In certain embodiments, the cationic agent is a growth factor. Preferably, the growth factor is VEGF, PDGF, FGF, bFGF, or HGH.

In certain embodiments, the cationic agent is at least one of VEGF and PDGF.

In certain embodiments, the anionic agent is a member selected from the group consisting of polysaccharides, glycosaminoglycans, complex carbohydrates, polyacids, modifications and derivatives thereof.

In certain embodiments, the anionic agent is a member selected from the group consisting of dextran, dextran sulfate, chitosan, heparin, heparan, heparan sulfate, hyaluronic acid, chondroitin, chondroitin sulfate, dermatan sufate, keratan sulfate, pentasan sulfate, alginate, and carageenan, polyglutamic acid, and 3-polyphosphoric acid.

In certain embodiments, the anionic agent is is dextran or dextran sulfate having a molecular weight of about 2 KDa to about 10,000 KDa.

In certain embodiments, the particle is free of poly(ethyleneimine).

Also provided is a particle consisting essentially of a complex between a growth factor and a polysaccharide.

Further provided is a method of making the particle of the invention, the method comprising: providing a bioactive agent; providing a complexing agent; and mixing the bioactive agent and the complexing agent at a pH of about 1 to about 13 to form the complex between the bioactive agent and the complexing agent, provided that the bioactive agent and the complexing agent are structural parts of the complex, and thereby forming the particle. Preferably, the pH is from 4.5 to 7.5.

In certain embodiments of the manufacturing method, the complex is formed by an electrostatic interaction between the bioactive agent and the complexing agent.

In certain embodiments of the manufacturing method, mixing is conducted in a low ionic strength buffer.

In certain embodiments of the manufacturing method, the buffer is an MES buffer.

In certain embodiments, the method further comprises adding a stabilizing agent selected from the group consisting of mono and disaccharides. Preferably, the stabilizing agent is selected from the group consisting of glucose, monose, trehalose, glycerol, and albumin.

In certain embodiments, the method further comprises providing at least one of a matrix and an agent.

In certain embodiments of the manufacturing method, the matrix is a member selected from the group consisting of biodegradable polymers, colloidal particles, liposomes, emulsions, solid particles, magnetic particles, proteins, and peptides and the agent is a member selected from the group consisting of an antibody, an antigen, a receptor, and a ligand.

Also provided is a method of administering of the particle of the invention, comprising:

providing the particle, wherein the particle is adapted to gradually release the bioactive agent; and administering the particle to a cell or an organism by at least one of a parenteral, an inhalation or an oral route.

In certain embodiments, administering is done by the parenteral route, for example, by an injection.

### BRIEF DESCRIPTION OF SEVERAL VIEWS OF THE DRAWINGS

The invention will be described in conjunction with the following drawings in which like reference numerals designate like elements and wherein:

FIG. 1A is a graph showing a particle size and loading stability (entrapment) of PDGF versus time.

FIG. 1B is a graph showing a change of a distribution of particle sizes with storage period. PDGF entrapment is expressed as a ratio of PDGF formulated as a particle to the total amount of PDGF in the preparation (1.0 mg/ml).

FIG. 2A is a graph demonstrating PDGF release into a medium without serum (NaCI/MES).

FIG. 2B is a graph demonstrating PDGF release into a simulated serum medium (albumin/NaCl/MS).

FIG. 3 is a graph demonstrating the effect of the particle formation time on the extent of the PDGF entrapment for the immediately formed preparation (0 hr) in comparison to the preparations obtained by 24 and 48 hr incubations at 4° C.

FIG. 4 is a bar graph showing a comparison of the bound and unbound PDGF in a particle formulation before (I) and after dialysis (II), wherein the unbound PDGF is shown as a white part and the bound PDGF is shown as a shaded part.

FIG. 5 is a photograph showing green (live cells) and red (dead cells) fluorescence staining of A10 cells to evaluate particles' toxicity to the cells. Cells were incubated with a PDGF-containing particles suspension (0.56 mg PDGF/ml) (I) in a cell growth medium (DMEM). Serum supplemented (20%) medium (II) and serum-free medium (III) were used as controls.

FIG. 6 is a bar graph showing results of a direct count of cells treated with PDGF-containing particles, native and denatured, and free PDGF, wherein the white area depicts denatured particles containing PDGF, the black area depicts native particles containing PDGF, and a grey area depicts free PDGF.

FIG. 7 is a graph showing results of WST-1 assay of cells treated with PDGF-containing particles, native and denatured, and free PDGF, wherein a square depicts denatured particles containing PDGF, a circle depicts native particles containing PDGF, and a diamond depicts free PDGF.

FIG. 8 is a photograph demonstrating results of Western-Blot electrophoresis on a 15% SDS-PAGE gel. The samples included free PDGF and PDGF particles applied at two amounts each (2 μl and 20 μl), one sample of PDGF released from the particles by the 8 day time point, and a PDGF ELISA kit standard (R&D Systems).

### DETAILED DESCRIPTION OF THE INVENTION

The invention was driven by a desire to prepare a new sustained release delivery system comprising a stable particle having a bioactive function. The particle of the invention comprises a complex between a bioactive agent and a complexing agent, wherein the bioactive agent is other than a polynucleotide and an oligonucleotide, and wherein the particle has a bioactive function conferred by the bioactive agent. The bioactive agent is gradually released in situ from the particle when administered to a cell or a tissue. The particle of the invention differs from the particles known in the art in that the bioactive agent is utilized as an essential structural part of the complex, i.e., the complex is formed due to the interactions between the bioactive agent and the complexing agent.

The particle of the invention is prepared by methods which do not require exposure to aggressive conditions while preserving the chemical stability and therapeutical potential of a bioactive gent or while conferring a new biological activity that resulted from of the association of the bioactive agent with the complexing agent.

The formulation method proposed herein differs from prior technologies because it allows preparation of particles capable of sustained release of a bioactive agent joined with a complexing agent, wherein the particles have a diameter in the 1 nm to 1000 micron size range and can be prepared using mild conditions and a broad range of pH from about 1 to about 13.

In certain embodiments, the invention is concerned with formulation of growth factors possessing the angiogenic effect beneficial in the treatment of myocardial infarction in injectable particles by the complex coacervation process employing mild conditions, thus allowing preserving the biological efficacy and minimizing toxic effects. Avoidance of harmful preparation conditions is especially important for biologically active proteins that are subject to considerable deactivation by many of the existing particle formulation methods.

Thus, the present invention has a number of beneficial features:
1. Stable particles in the size range of from about 1 nm to about 1000 microns;
2. Fast and reproducible methods of making particles;
3. Use of biocompatible substances and avoidance of potentially harmful preparation conditions;
4. Use of auxiliary agents contributing to minimization of toxicity and potentiation of the therapeutic efficacy.

Particles of the invention can deliver bioactive agent to a cell or an organism via a parenteral, an inhalation or an oral route, wherein a depot of particles slowly releases the therapeutic substance. Preferably, the delivery is by a parenteral route (i.e., an injection) including parenterall, intravenously and intramuscularly. A mixture of different sizes can also be used.

In one of the embodiments, the bioactive agent is an angiogenic growth factor stimulating angiogenesis into myocardial tissue (or other sites requiring revascularization). This is expected to prevent rapid escape of the growth factors from the target tissue and provide the sustained presence of the growth factor for a therapeutically effective time period.

The particles of the invention will be useful in various medical applications, for example, for diabetes (insulin), bone regeneration (BMP's), cancer therapy (Fas-L), and angiogenesis (VEGF and PDGF).

Particle of the Invention

The terms “microparticle”, “nanoparticle”, and “particle” are used interchangeably in this disclosure and collectively describe a particle in a 1 nm to 1000 micron size range, wherein a microparticle has a diameter in a range of between 1 to 1000 microns and a nanoparticle has a diameter of less than 1 micron up to and including 1 nm. Depending on the route of administration, the desired size can be selected, for example, microparticles having at least 15 microns in size can be used for topical or oral delivery, microparticles having 1-5 micron size range can be used for inhalation, and parenteral administration generally requires particles below 5 microns and preferably below 1 micron. These numbers are not limiting and shown herein as examples. The size of particles can be varied based on a method of preparation, the nature and amounts of components used as well as conditions such as pH and temperatures.

The particle of the invention comprises a complex between a bioactive agent and a complexing agent, wherein the bioactive agent is other than a polynucleotide and an oligonucleotide, and wherein the particle has a bioactive function conferred by the bioactive agent. The bioactive agent is gradually released in situ from the particle when it is administered to a cell or a tissue. In the particle of the invention, the bioactive agent is utilized not as an additive to a particle but as an essential structural part of the complex, i.e., the complex is formed due to the interactions between the bioactive agent and the complexing agent. The term “entrapment” is used herein to indicate that the bioactive agent is “entrapped” by the complexing agent in a form of a particle.

In certain embodiments, the particle of the invention is formed by an ionic interaction between a cationic agent and an anionic agent, wherein at least one of the above agents is bioactive. In certain embodiments, the biomolecule and the complexing agent have an opposite charge.

Bioactive Agent

Non-limiting examples of bioactive agents of the invention are proteins such as growth factors (e.g., VEGF, PDGF, FGF, bFGF, HGH), erythropoietin (EPO), extracellular superoxide dismutase, the bone morphogenic proteins (BMP's), human chorionic gonadotrophin, hormones such as insulin, peptides such as cell penetrating peptides, complex carbohydrates such as polysaccharides (e.g., chitosan), receptor sequences (e.g., receptor fragments for FGF, PDGF, VEGF, CAR, transferrin, TGF-beta receptors, and all the integrin heterodimer receptor family). Bioactive agents can have a naturally occurring, synthetic or recombinant origin.

The term “therapeutic activity” as used herein includes, for example, an activity such as prevention or alleviation of a condition such as, for example, angiogenesis (VEGF, PDGF), diabetes (insulin), bone regeneration (BMP's), and cancer therapy (Fas-L). Depending on a choice of the bioactive agent, the particle of the invention has a therapeutic activity for a desired application such as, for example, angiogenesis (VEGF, PDGF), diabetes (insulin), bone regeneration (BMP's), and cancer therapy (Fas-L).

The term “biological activity” or “bioactive function” as used herein means an activity or a function conferred by the bioactive agent of choice as well as an activity or a function conferred by a formation of the particle using the bioactive agent with the complexing agent of choice. Formation of the particle can influence/modify a degree/nature of the desired biological activity. However, the biological activity of the bioactive agent is preferably preserved after formation of the complex.

In certain embodiments, the bioactive agent has a net positive charge or has a positively charged region of at least +6 or a negative net negative charge or the negatively charged region of at least −6. Preferably, the bioactive agent is positively charged.

In certain embodiments, the net positive charge is conferred by at least six amino acids selected from the group consisting of lysine, arginine, and histidine.

In certain embodiments, the positively charged region is a heparin binding domain.

The term “heparin binding domain” as used herein is a region of amino acids capable of binding heparin and having a positive charge conferred by the presence of argentine, lysine and histidine. Examples of the heparin binding domain are known in the art and are stated above.

In the present invention, the charge can be also conferred by chemical modification and covalent bonding of polar chemical groups.

Complexing Agent

A complexing agent is a polyelectrolyte, which is preferably charged oppositely to a biomolecule, wherein the charge is on the whole molecule or in part of the molecule.

In certain embodiments, the complexing agent has the net negative charge or the negatively charged region of at least −6 or the net positive charge or the positively charged region of at least +6. Preferably, the complexing agent is negatively charged.

Non-limiting examples of complexing agents are polysaccharides such as dextran, sulfated polysaccharides such as dextran sulfate, chitosan, glycosaminoglycans (GAG) such as heparin, heparan, heparan sulfate, hyaluronic acid, chondroitin, chondroitin sulfate, dermatan sufate, keratan sulfate, and pentasan sulfate, complex carbohydrates such as alginate and carageenan, polyacids such as polyglutamic acid, 3-polyphosphoric acid, and modifications and derivatives thereof. Preferably, the complexing agent is dextran or dextran sulfate having a molecular weight of about 2 KDa to about 10, 000 KDa, and more preferably, 5-500 KDa. Complexing agents can have a naturally occurring, synthetic or recombinant origin.

The complexing agent can be a cationic agent or an anionic agent. As used herein, “a cationic agent” is a biodegradable heteropolymer bearing a cationic region or having a positive net charge sufficient to bind an anionic agent. The term “heteropolymer” as used herein means that some of monomers of the heteropolymer are negatively charged, however, the overall charge of the heteropolymer is positive. The heteropolymer includes polymeric complexes of poly(hydroxy acids) with, for example, poly(ethyleneimine) or polyallylamine.

The cationic agent can be a biomolecule having a desired therapeutic activity or a molecule without therapeutic activity. Cationic agents can have a naturally occurring, synthetic or recombinant origin.

Non-limiting examples of cationic agents having a desired therapeutic activity are growth factors such as VEGF, PDGF, hormones such as insulin, peptides, proteins, and complex carbohydrates, e.g., polysaccharides such as chitosan.

Non-limiting examples of cationic agents without therapeutic activity are proteins such as histone, protamine, spermidine, and spermine.

As used herein, “an anionic agent” is a polymer (a heteropolymer or a homopolymer) bearing a negative net charge, optionally having a desired therapeutic activity.

Non-limiting examples of anionic agents without therapeutic activity are dextran, dextran sulfate, heparin, glycosaminoglycans (GAG) and their component polysaccarides (heparin, heparan, hyaluronic acid, and chondroitin sulfate), alginate, carageenan, and synthetic polybisphosphonates such as polyallylamine bisphosphonates.

Non-limiting examples of anionic agents with therapeutic activity are heparin, low molecular weight heparin, poly(glutamate), and poly-N-arylaminoacid.

Anionic agents can be naturally occurring, synthetic or recombinant.

Particles Variants

In certain embodiments, particles of the invention represent an ionic pair, wherein either the cationic agent or the anionic agent or both consist of a bioactive agent having a desired therapeutic activity. Non-limiting examples of such particles are PDGF/dextran, PDGF/dextran sulfate, VEGF/dextran, and EPO/dextran.

Further, particles of the invention may include an additional agent for various functions such as, for example, a stabilizing function wherein the particles will be protected against degradation by a cross-linking agent or another agent, a targeting function, wherein various ligands are included to interact with certain cells, an efficiency enhancing function by utilizing receptors to biomolecules used to form particles, and a potential toxic effect reducing function by balancing concentrations of particles in cells.

Additionally, the particles of the invention can be labeled for detecting purposes using various labels and methods known to those skilled in the art.

The particles of the invention can also include an agent adapted to impart an additional biological function. A non-limiting example of the agent is an antibody, an antigen, a receptor, and a ligand.

Non-limiting examples of additional agents are liposome disruptive agents, nuclear localization sequences, cell penetrating peptides, and morphine sulfate.

In certain embodiments of the invention, the particles of the invention can further associate with a matrix, which may or may not have a therapeutic activity. Non-limiting examples of such matrix are biodegradable polymers (e.g., polyhydroxyacids), colloidal particles, liposomes, emulsions, solid particles, magnetic particles, proteins, and peptides. Association of the particles of the invention with the matrix can be based on, for example, electrostatic interaction, adsorption, adhesion, affinity (antibody-antigen, receptor-ligand) or covalent bonding. In that, the matrix can be added to the particles at any stage of the formation of either the matrix or the particles.

For example, a complex between a PDGF/dextran, or PDGF/dextrane sulfate particle or PLGA particles can be formed wherein PDFG is first adsorbed onto PLGA particle surface and then mixed with dextran or dextran sufate.

Methods of making such matrixes are known in the art. For example, colloidal particle formation may be accomplished either by in situ polymerization of monomers either in aqueous solution or emulsified in aqueous phase (namely, emulsification-polymerization). Alternatively, methods exploiting pre-formed biocompatible polymers, usually of polyester and polyanhydride families, can be used to form particles by polymer precipitation methods. The most popular methods are emulsification-solvent evaporation, emulsification-diffusion and nanoprecipitation methods (See Quintanar-Guerrero D, Allémann E, Fessi H, Doelker E. Preparation techniques and mechanisms of formation of biodegradable nanoparticles from preformed polymers. Drug Dev Ind Pharm 1998; 24:1113-28). The latter methods are based on emulsifying an organic solution of a polymer with or without drug in an aqueous phase in presence of a stabilizer substance (e.g. POLOXAMER 188, polyvinyl alcohol, etc.) achieved either by an external energy input or through spontaneous diffusion of water-miscible solvents with subsequent solvent elimination to form solid particle dispersion.

In certain embodiments of the invention, the particles of the invention can further associate with an additional bioactive agent such as, for example cell penetrating peptides.

Preparation of Particles

The formation of the complexes is achieved by mixing aqueous solutions of a bioactive agent and a complexing agent (e.g., a polyelectrolyte bearing positive and negative charge, respectively) under the employed conditions (pH of about 1 to about 13, preferably, the pH is from 4.5 to 7.5 and more preferably pH 5.5-6.0). The mixture spontaneously associates into electrostatically stabilized ion-pair particles sized from about 1 nm to about 1000 microns, as described in the Example 1 below, forming a particle suspension. Preferably, the size of particles is in the range between 15 nm and 100 μm, and more preferably, 150-220 nm. The size of particles can vary depending on the choice of reagents, their amounts and operating conditions such as temperature and pH. The obtained particle suspension can then be filtered through, for example, a six-micron paper filter to remove aggregates.

The particles can be used as is or upon dilution (preferably with low ionic strength media, such as glucose 5% solution in MES buffer (pH 6.0), 0.001 M). Optionally, the particles can be freeze-dried and stored for later use and then used after re-suspension in purified water or another suitable medium.

Without being bound by a specific theory, the inventors acknowledge that formation of the particle of the invention is potentially based on a complex coacervation, however other mechanisms such as the thermodynamic incompatibility, hydrogen bonding or hydrophobic interactions as described by Turgeon et al. are also possible herein. Moreover, the complexation between a polyelectrolyte and a protein may still occur even when both macromolecules are negatively charged.

In certain embodiments, the method further comprises adding a stabilizing agent selected from the group consisting of mono- and disaccharides. Preferably, the stabilizing agent is selected from the group consisting of glucose, monose, trehalose, glycerol, and albumin.

Also provided is a method of administering of the particle of the invention, comprising providing the particle, wherein the particle is adapted to gradually release the bioactive agent; and administering the particle to a cell by at least one of a parenteral, an inhalation or an oral route. Preferably, administering is done by the parenteral route.

The invention will be illustrated in more detail with reference to the following Examples, but it should be understood that the present invention is not deemed to be limited thereto.

### EXAMPLES

### Example 1

Method of Making Particles

Solution A: dextran sulfate was dissolved in purified water in concentration 1% w/v.

Solution B: PDGF-BB was dissolved in aqueous solution of glucose (5% w/v) to yield 1 mg/ml; pH was adjusted to 6.0 by acetate buffer (0.005% w/v) or MES buffer (0.001 M).

Solution A was added dropwise to solution B on magnetic stirring to the final volume ratio 100-150 μl:10 ml. The obtained particle suspension was filtered through 6 μm paper filter to remove aggregates.

The PDGF-binding particles can be used as is or upon dilution (preferably with low ionic strength media, such as glucose 5% solution in MES buffer (pH 6.0), 0.001 M). Optionally, the particles can be stored freeze-dried and used after re-suspension in purified water or another suitable medium. The particle preparation maybe accomplished under sterile conditions yielding a ready-to-use product or sterilized by a suitable method (irradiation, sterile filtration) prior to injecting.

### Example 2

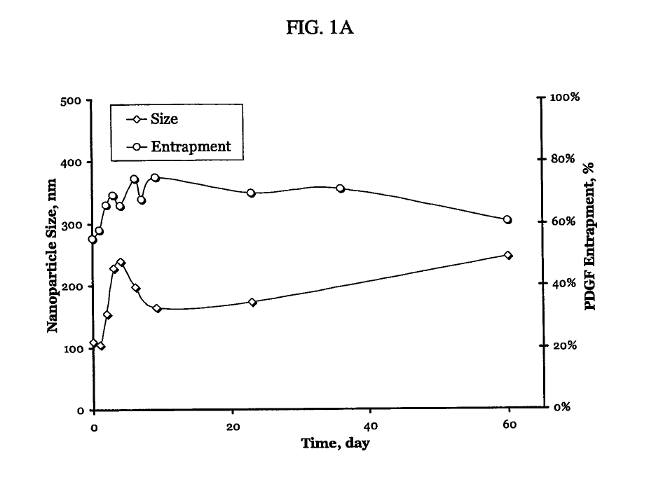

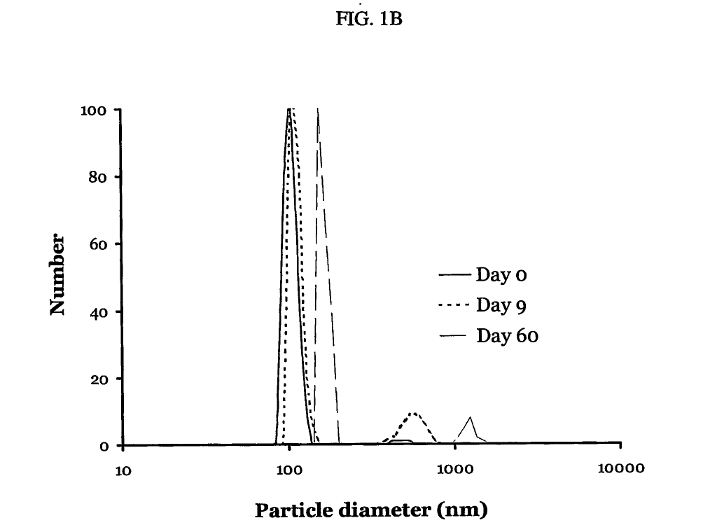

PDGF/dextrane sulfate particles prepared as described in Example 1 were studied for size and entrapment/loading as shown in FIG. 1A. FIG. 1A demonstrates efficient entrapment and a uniform (200-250 nm range) particle size.

Labeling of the bioactive agent (PDGF) was conducted as follows: 3.0 mg of carboxytetramethylrhodamine N-hydroxysuccinimidyl ester was mixed with 4.0 ml PDGF-BB solution (10 mg/ml, acetate buffer pH 6.2), and 900 μl of bicarbonate buffer (pH 8.5, 0.1 M). The mixture was stirred for 5 minutes on a vortex, and placed in the dark at room temperature for 1 hr, then dialyzed overnight (molecular weight cut off 2000 KDa) in 1 L of acetate buffer (pH 6.0, 0.005%).

Size determination was conducted as follows: 60 μl of particle suspension were added to 3.0 ml of MES buffer (pH 6.5, 0.001 M), and their size measurement was performed by photon correlation spectroscopy for 2 minutes and presented as a number-weighted size distribution. Size stability was determined for coacervate particles kept at 4° C. during a 60 days period. Measurements show gradual increase of the particle size in the main population and formation of a second larger-sized population over time (FIG. 1B).

Loading stability was measured over a 60 days period for the particle preparation stored at 4° C. The entrapment at each given time point was obtained from the fluorescent signal of the complete particle suspension and the fluorescent signal in suspending medium separated by particles centrifugation. % Entrapment was calculated using the formula below:

$$\% Entrapment = \frac{Total - Suspending\_medium}{Total} \times 100 \%$$

The minimal signal interference was observed for a non-fluorescent particles suspension in a control experiment. Measurements demonstrate entrapment of 60-75% of PDGF in particles that remains stable during the studied storage period of 60 days (FIG. 1A).

### Example 3

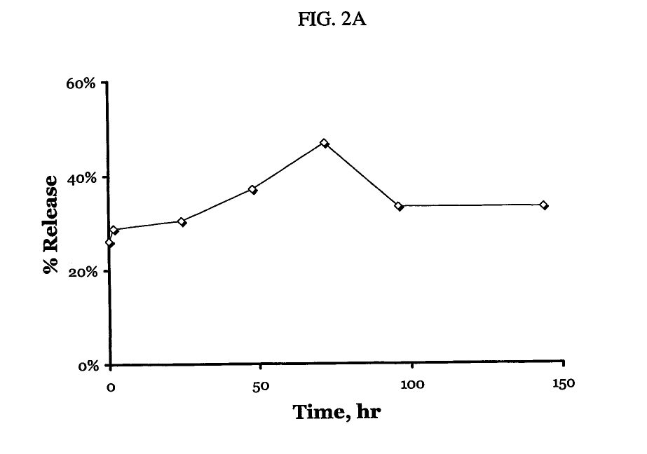

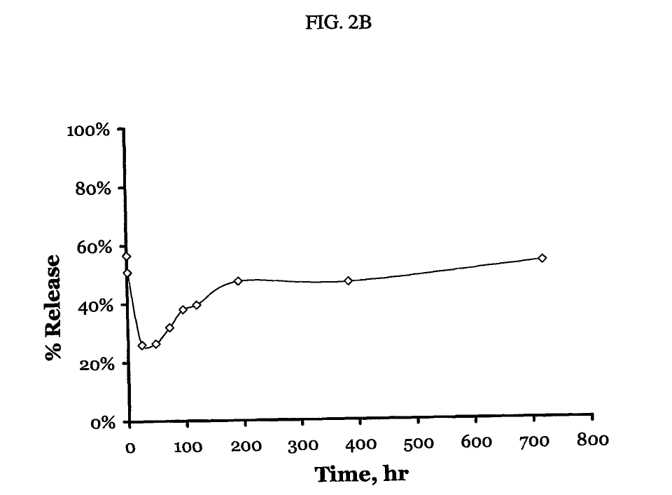

Dextran sulfate-PDGF particles were prepared as described in Example 1. The preparation was examined for release kinetics. A release medium was prepared in a sterile fashion by (a) adding 0.5 ml MES buffer (pH 6.5, 0.1 M) to 50 ml saline (0.9% NaCl in deionized water) (see FIG. 2A) and (b) adding 0.5 ml MES buffer (pH 6.5, 0.1 M) to 50 ml bovine serum albumin/saline (5% albumin and 0.9% NaCl in deionized water) (see FIG. 2B). 2.5 ml of the particles were mixed with the release medium, transferred into a 60-ml syringe equipped with a 0.02 μm sterile filter. The syringe was placed on a LABQUAKE shaker (Apogent Technologies, Inc., Portsmouth, N.H.) at room temperature in the dark. At predetermined time points, a 100 μl sample of the release medium was filtered following extraction of the first 10 drops to be discarded. The amount of fluorescently labeled protein was determined in comparison to a sample of particle suspension in the release medium, which was kept in the dark at room temperature as a reference.

Under the examined conditions, more than 40% of PDGF initially present in the preparation remained unreleased in a serum free medium. A higher release was observed in the albumin containing release medium.

### Example 4

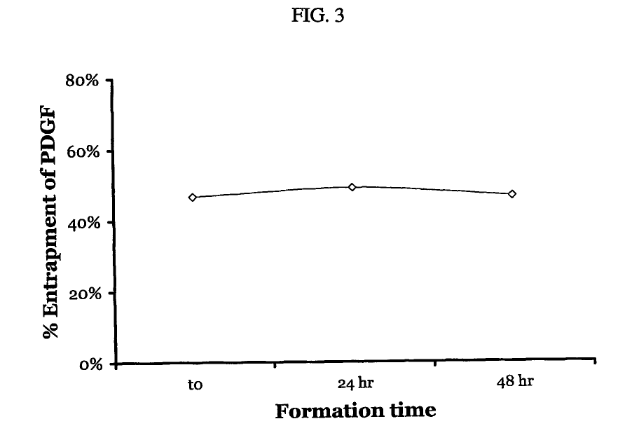

The effect of particle formation time on PDGF entrapment was evaluated in an immediately formed preparation in comparison to preparations obtained by 24 and 48 hr incubation at 4° C. (FIG. 3). The particle formation time appears to have no effect on % entrapment.

### Example 5

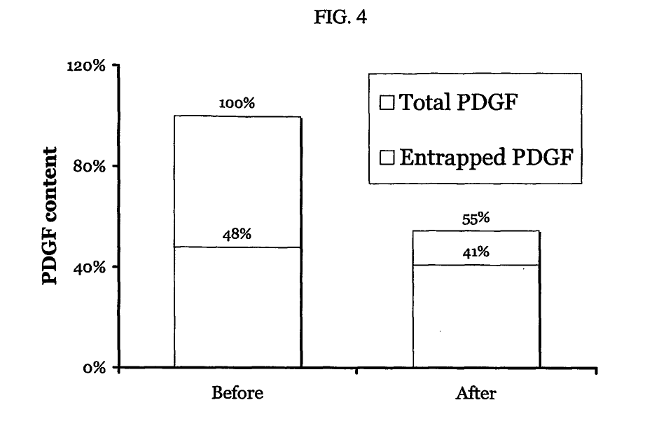

Dialysis was evaluated as a strategy for elimination the particle unbound PDGF (see FIG. 4). The particle suspension was sealed in a dialysis membrane and dialyzed for 24 hours at 4° C. against MES buffer (pH 6.5, 0.001 M) containing 5% glucose using a 300 KDa cut-off membrane. It was observed that while the total amount of PDGF decreased to 55% of the initial concentration, the amount of the unbound protein was reduced by almost four-fold corresponding to about 25% of the total PDGF concentration in the dialyzed preparation as opposed to 52% in the initial preparation. The dialysis also reduced the amount of particle bound protein by about 15%.

### Example 6

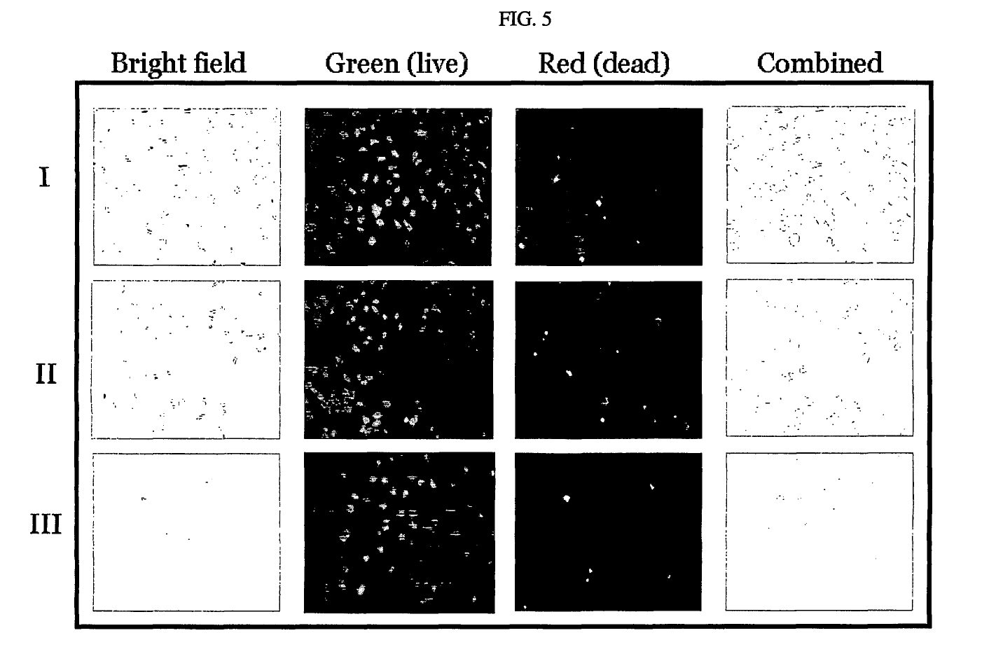

In this experiment, particles' toxicity to smooth muscle cells (A10) was evaluated. Rat aortic smooth muscle cells (A10) were seeded on a 96-well plate at a density of 5×10³/well. Cells were incubated with PDGF-containing particle suspension (0.56 mg PDGF/ml) (I) in a cell growth medium (DMEM) (see FIG. 5). Serum supplemented (20%) medium (II) and serum-free medium (III) were used as controls. After 4 days of incubation, the cells were stained with calcein and ethidium homodimer as described by the manufacturer (Molecular Probes Inc., Eugene, Oreg.) and their green and red fluorescence (live and dead cells, respectively) was observed using fluorescent microscopy. The amount of live cells in the particle treated group (I) was not reduced as compared to the serum-supplemented medium control group (II), whereas fewer cells were observed in the serum-free medium group (III). Additionally, particle treatment did not result in a more extensive cell death as compared to the control groups.

### Example 7

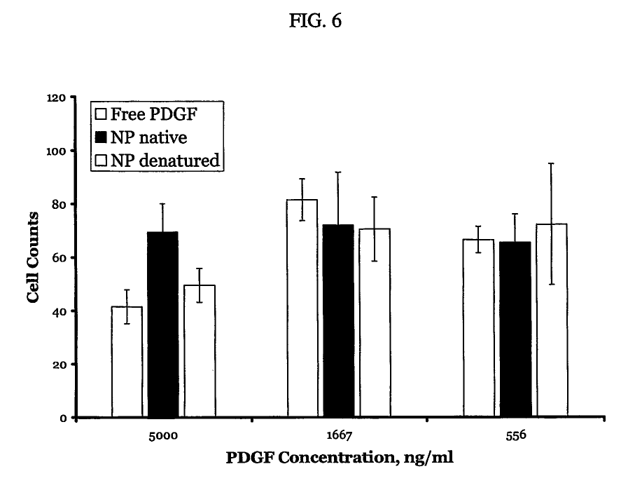

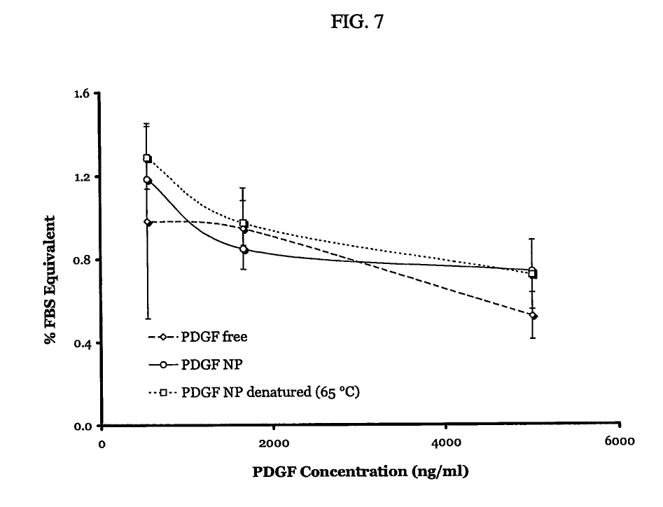

Cell mitogenic effect of PDGF-containing particles was evaluated in comparison to free PDGF and PDGF-containing particles subjected to 15 min-treatment with heat (65° C.). Dilutions of particles and free PDGF were made in a cell growth medium (DMEM) and applied to A10 cells seeded on the previous day at a density of 2×10³/well on the 96-well plates. The effect was evaluated after 1 day of incubation by the direct cell counting (FIG. 6) and by WST-1 cell proliferation assay (FIG. 7) using the protocol as described by the manufacturer (Roche, Indianapolis, Ind.). Particles with or without heat treatment show a proliferative effect similar to that of free PDGF. Complexation of PDGF into coacervate particles appears to have a protective effect, i.e., preventing irreversible denaturation of PDGF based on the observed mitogenic effect which is not inferior to that of the untreated particles. WST-1 assay is a calorimetric assay for quantification of cytotoxicity, based on cleavage of the WST-1 tetrazolium salt by mitochondrial dehydrogenases in viable cells.

### Example 8

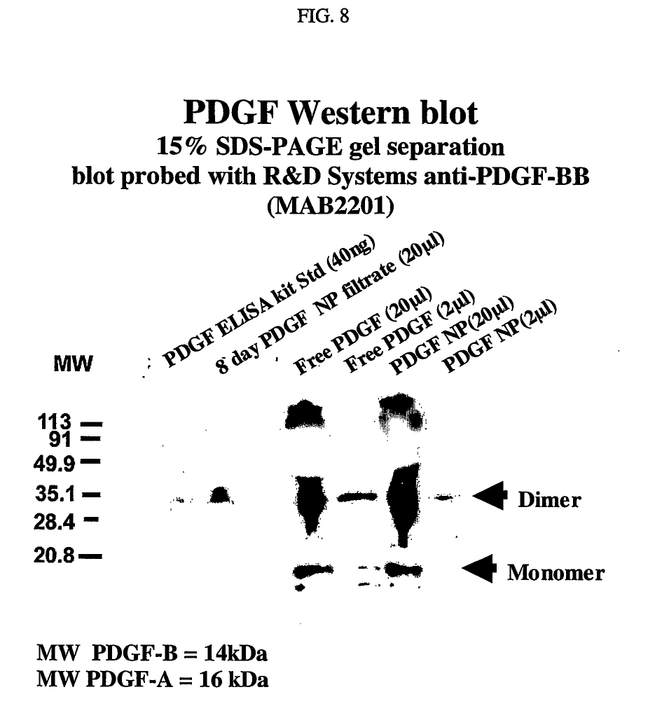

Particles prepared as described in Example 1 were analyzed by Western-Blot electrophoresis, wherein the separation was made using 15% SDS-PAGE gel according to the established procedure (see FIG. 8). The samples included free PDGF and PDGF particles applied at two amounts each (2 μl and 20 μl), one sample of PDGF released from the particles by the 8-day time point, and a PDGF ELISA kit standard (R&D Systems). The results demonstrated retained structure and stability of PDGF incorporated in the particles, as well as released from the latter.

While the invention has been described in detail and with reference to specific examples thereof, it will be apparent to one skilled in the art that various changes and modifications can be made therein without departing from the spirit and scope thereof.

What is claimed is:

1. A particle comprising a complex formed by a bioactive agent and a complexing agent, provided that the bioactive agent is other than a polynucleotide and an oligonucleotide, and wherein the particle has a bioactive function conferred by the bioactive agent.

2. The particle of claim 1, wherein the bioactive agent and/or the complexing agent have a net positive charge or have a positively charged region of at least +6.

3. The particle of claim 1, wherein the bioactive agent has the net positive charge or the positively charged region of at least +6 and the complexing agent has the net negative charge or the negatively charged region of at least −6.

4. The particle of claim 1, wherein the net positive charge is conferred by at least six amino acids selected from the group consisting of lysine, arginine, and histidine.

5. The particle of claim 2, wherein the positively charged region is a heparin binding domain.

6. The particle of claim 2, wherein the other of the bioactive agent and the complexing agent has a net negative charge or has a negatively charged region of at least −6.

7. The particle of claim 1, wherein the particle has a diameter from about 1 nm to about 1000 microns.

8. The particle of claim 1, wherein the bioactive agent is a member selected from the group consisting of a growth factor, a hormone, a peptide, a protein, and polysaccharide.

9. The particle of claim 1, wherein the bioactive agent is a growth factor.

10. The particle of claim 1, wherein the growth factor is VEGF, PDGF, FGF, bFGF, or HGH.

11. The particle of claim 1, wherein the bioactive agent is at least one of VEGF and PDGF.

12. The particle of claim 1, wherein the bioactive agent is a member selected from the group consisting of insulin, erythropoietin, bone morphogenic proteins, human growth hormone, human chorionic gonadotrophin, polysaccharides, transferrin, TGF-beta receptors, integrin heterodimer receptor, Fas-L, and receptor fragments for FGF, PDGF, VEGF, and CAR.

13. The particle of claim 1, wherein the complexing agent is a member selected from the group consisting of polysaccharides, glycosaminoglycans, complex carbohydrates, polyacids, modifications and derivatives thereof.

14. The particle of claim 1, wherein the complexing agent is a member selected from the group consisting of dextran, dextran sulfate, chitosan, heparin, heparan, heparan sulfate, hyaluronic acid, chondroitin, chondroitin sulfate, dermatan sufate, keratan sulfate, pentasan sulfate, alginate, and carageenan, polyglutamic acid, and 3-polyphosphoric acid.

15. The particle of claim 14, wherein the complexing agent is dextran or dextran sulfate having a molecular weight of about 2 KDa to about 10,000 KDa.

16. The particle of claim 15, wherein the molecular weight of dextran or dextran sulfate is from 5 KDa to 500 KDa.

17. The particle of claim 1, wherein the particle is free of poly(ethyleneimine).

18. The particle of claim 1, further comprising an agent, wherein the agent is a member selected from the group consisting of an antibody, an antigen, a receptor, and a ligand.

19. The particle of claim 1, further comprising a matrix, wherein the matrix is associated with at least one of the bioactive agent and the complexing agent.

20. The particle of claim 19, wherein the matrix is a member selected from the group consisting of biodegradable polymers, colloidal particles, liposomes, emulsions, solid particles, magnetic particles, proteins, and peptides.

21. The particle of claim 20, wherein the particle is free of poly(ethyleneimine).

22. The particle of claim 1, wherein the particle is completely biodegradable.

23. The particle of claim 1, wherein the particle comprises at least 40% of the bioactive agent.

24. The particle of claim 1, wherein the particle comprises from about 40% to about 90% of the bioactive agent.

25. The particle of claim 1, wherein the particle consists essentially of the bioactive agent and the complexing agent.

26. A particle comprising a complex formed by a bioactive agent and a complexing agent, wherein one of the bioactive agent and the complexing agent is a cationic agent or an anionic agent having a net charge or a region having a net charge of at least 6 units and wherein the particle has a bioactive function conferred by the bioactive agent.

27. The particle of claim 26, wherein the bioactive agent is the cationic agent and the complexing agent is the anionic agent.

28. The particle of claim 26, wherein the cationic agent is a member selected from the group consisting of a growth factor, a hormone, a peptide, a protein, and polysaccharide.

29. The particle of claim 28, wherein the cationic agent is a growth factor.

30. The particle of claim 29, wherein the growth factor is VEGF, PDGF, FGF, bFGF, or HGH.

31. The particle of claim 1, wherein the cationic agent is at least one of VEGF and PDGF.

32. The particle of claim 26, wherein the anionic agent is a member selected from the group consisting of polysaccharides, glycosaminoglycans, complex carbohydrates, polyacids, modifications and derivatives thereof.

33. The particle of claim 26, wherein the anionic agent is a member selected from the group consisting of dextran, dextran sulfate, chitosan, heparin, heparan, heparan sulfate, hyaluronic acid, chondroitin, chondroitin sulfate, dermatan sufate, keratan sulfate, pentasan sulfate, alginate, and carageenan, polyglutamic acid, and 3-polyphosphoric acid.

34. The particle of claim 31, wherein the anionic agent is is dextran or dextran sulfate having a molecular weight of about 2 KDa to about 10,000 KDa.

35. The particle of claim 26, wherein the particle is free of poly(ethyleneimine).

36. A particle consisting essentially of a complex between a growth factor and a polysaccharide.

37. A method of making the particle of claim 1, the method comprising:
providing a bioactive agent;
providing a complexing agent; and
mixing the bioactive agent and the complexing agent at a pH of about 1 to about 13 to form the complex between the bioactive agent and the complexing agent, provided that the bioactive agent and the complexing agent are structural parts of the complex, and thereby making the particle.

38. The method of claim 37, wherein the pH is from 4.5 to 7.5.

39. The method of claim 37, wherein the complex is formed by an electrostatic interaction between the bioactive agent and the complexing agent.

40. The method of claim 37, wherein mixing is conducted in a low ionic strength buffer.

41. The method of claim 40, wherein the buffer is an MES buffer.

42. The method of claim 37, further comprising a stabilizing agent selected from the group consisting of mono and disaccharides.

43. The method of claim 40, wherein the stabilizing agent is selected from the group consisting of glucose, monose, trehalose, glycerol, and albumin.

44. The method of claim 37, further comprising providing at least one of a matrix and an agent.

45. The method of claim 37, wherein the matrix is a member selected from the group consisting of biodegradable polymers, colloidal particles, liposomes, emulsions, solid particles, magnetic particles, proteins, and peptides and wherein the agent is a member selected from the group consisting of an antibody, an antigen, a receptor, and a ligand.

46. A method of administering of the particle of claim 1, comprising:
providing the particle, wherein the particle is adapted to gradually release the bioactive agent; and
administering the particle to a cell by at least one of a parenteral, an inhalation or an oral route.

47. The method of claim 46, wherein administering is done by the parenteral route.
